(19) United States
(12) Patent Application Publication
(10) Pub. No.: US 2018/0197041 A1
(43) Pub. Date: Jul. 12, 2018

(54) Service Providing Using An Image

(71) Applicant: UIPCO, LLC, San Antonio, TX (US)

(72) Inventors: Rachel Ann Krebs, San Antonio, TX (US); Jennifer Lynne Gravely, San Antonio, TX (US); Elizabeth Ann Guerrero, San Antonio, TX (US)

(21) Appl. No.: 15/850,125

(22) Filed: Dec. 21, 2017

Related U.S. Application Data
(60) Provisional application No. 62/443,126, filed on Jan. 6, 2017.

(51) Int. Cl. G06K 9/62

(52) U.S. Cl. CPC G06K 9/62; G06K 9/00362

(57) ABSTRACT

Methods and apparatus to receive and process an image for the provision of services based on objects identified in the image. In one embodiment, the objects include at least one person and the services includes medical information for an injury to the person, insurance based on age of the person. In another embodiment, the objects include at least one structure and the services includes repair and/or maintenance for the structure.

### CROSS REFERENCE TO RELATED APPLICATIONS

The present application claims the benefit of U.S. Provisional Patent Application No. 62/443,126, filed on Jan. 6, 2017, which is incorporated herein by reference.

### SUMMARY

In one aspect, a method comprises: receiving an image; processing the image to identify one or more objects in the image; and selecting a service based on the one or more objects identified in the image. A method can further include one or more of the following features: the image is a photo, the image is a video, the one or more objects includes a person and the service is selected based upon an age of the person, the one or more objects includes at least two people and the service is selected based upon an identified relationship between the at least two people, the one or more objects includes a person with a disability and the service is selected based upon the disability, the one or more objects includes medical injury and the service is selected based upon the injury, the one or more objects includes food and the service is selected based upon a type of the food, the one or more objects includes food and the service is selected based upon a type of the food for food allergy detection, the one or more objects includes cooked meat and the service is selected based upon an identified doneness of the meat, the one or more objects includes a structure and the service is selected based damage to the structure, the one or more objects includes a structure and the service is selected based upon maintenance needed for the structure, the one or more objects includes a structure and the service is selected based upon maintenance needed for the structure, wherein the service includes a cost estimate for the needed maintenance, the service includes shipping of the one or more objects and the service is selected based upon an estimate weight of the one or more objects, the service includes shipping of the one or more objects and the service is selected based upon an estimate weight of the one or more objects, and the service includes a cost estimate for shipping of the one or more objects, and/or the one or more objects includes objects in a home and the service includes insurance services for protecting against loss or damage to the one or more objects.

In another aspect, a system comprises: an image module to receive an image; an image analyzer module for processing the image to identify one or more objects in the image; and a service module coupled to the image analyzer module for selecting a service based on the one or more objects identified in the image. A system can further include one or more of the following features: the image is a photo, the image is a video, the one or more objects includes a person and the service is selected based upon an age of the person, the one or more objects includes at least two people and the service is selected based upon an identified relationship between the at least two people, the one or more objects includes a person with a disability and the service is selected based upon the disability, the one or more objects includes medical injury and the service is selected based upon the injury, the one or more objects includes food and the service is selected based upon a type of the food, the one or more objects includes food and the service is selected based upon a type of the food for food allergy detection, the one or more objects includes cooked meat and the service is selected based upon an identified doneness of the meat, the one or more objects includes a structure and the service is selected based damage to the structure, the one or more objects includes a structure and the service is selected based upon maintenance needed for the structure, the one or more objects includes a structure and the service is selected based upon maintenance needed for the structure, wherein the service includes a cost estimate for the needed maintenance, the service includes shipping of the one or more objects and the service is selected based upon an estimate weight of the one or more objects, the service includes shipping of the one or more objects and the service is selected based upon an estimate weight of the one or more objects, and the service includes a cost estimate for shipping of the one or more objects, and/or the one or more objects includes objects in a home and the service includes insurance services for protecting against loss or damage to the one or more objects.

### BRIEF DESCRIPTION OF THE DRAWINGS

FIG. 1 is a simplified block diagram of one example of a system to provide a service using an image.

FIG. 2 is a flowchart of an example of a process to provide a service using the image.

FIG. 3 is a computer on which any portion of the process of FIG. 2 may be implemented.

### DETAILED DESCRIPTION

Described herein are techniques to provide a service based on an image. In one example, the image is a photo. In another example, the image is a video. In one example, the image is presented in lieu of filling out a form.

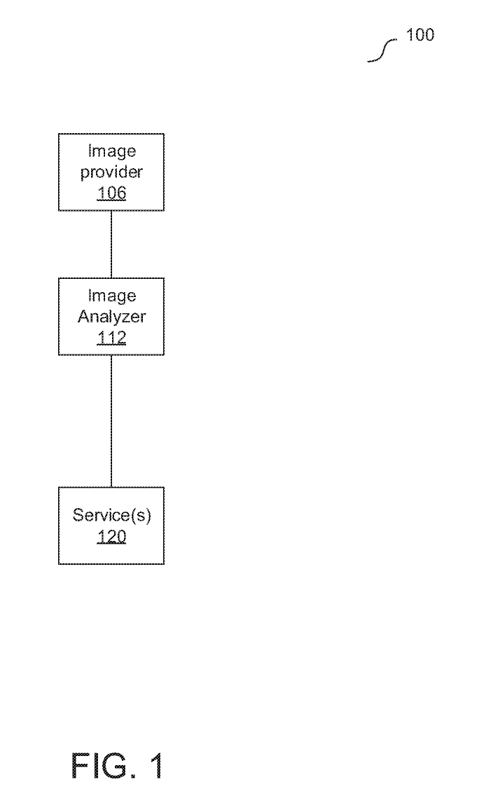

Referring to FIG. 1, a system 100 is an example of a system to provide a service using an image. The system 100 includes an image provider 106, an image analyzer 112 and one or more services. The image provider 106 provides an image (e.g., a photo or a video). In some examples, the image provider 106 may be a mobile device that includes a camera for taking photos and videos or a personal computer that includes photos and videos. In other embodiments, the image provider 106 comprises a database of image, videos, etc, for analysis by the image analyzer 112.

The image analyzer 112 analyzes the image received. For example, the image analyzer 112 searches for certain objects in the image. For example, the image analyzer 112 searches for objects that could be persons or things or both. The image analyzer 112 identifies one or more objects. For example, using facial recognition and/or object recognition programs, the image analyzer 112 may identify age and/or gender of one or more persons in the photo. In other examples, the image analyzer 112 identifies the type of objects.

The one or more services 120 are provided based on the one or more objects identified in the image. In one example, a service may be at least one of a financial service, a banking service, an insurance service or a health service.

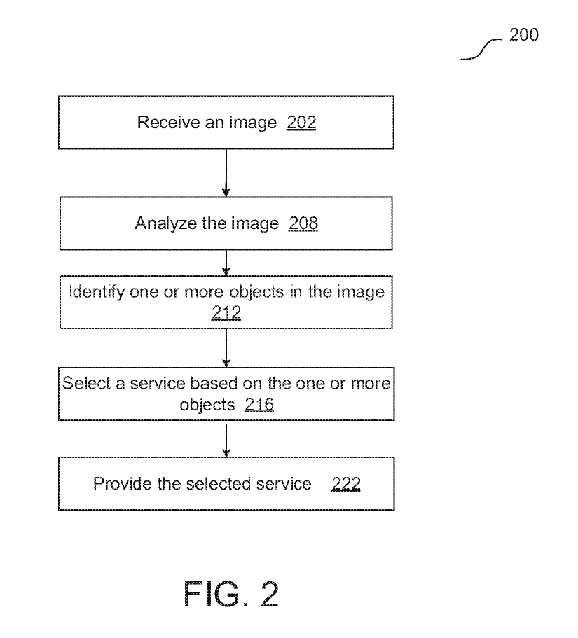

Referring to FIG. 2, an example of a process to provide a service using an image is a process 200. In one example, the image analyzer 112 performs the process 200. Process 200 receives an image (202). For example, an image file is uploaded to the image analyzer 112 using an image provider 106 (FIG. 1) that is a mobile device. In one example, a user is prompted to take a photograph of their family.

Process 200 analyzes the image (208). For example, the image analyzer 112 analyzes the image to locate one or more objects in the image. In one example, an image of three people is recognized in the image and an inanimate object.

Process 200 identifies one or more objects in the image (212). For example, the image analyzer 112 identifies the one or more objects. The image analyzer 112 determines that an older person is female and most likely the mother while the two younger males are her children. The system 100 identifies that one of the sons is in a wheelchair.

Process 200 selects a service based on the one or more objects (216) and process 200 provides the service (222). For example, the image analyzer 112 selects from the service(s) 120. In one particular example, the image analyzer 112 provides at least one of home insurance, life insurance, health insurance, financial services, and banking services options based on a family situation. For example, a first one of the one or more objects is identified as an automobile and a selected service comprises automobile insurance. If the make, model and year of the automobile is identified, the automobile insurance service can include a cost estimate for the insurance service.

The techniques described herein may also be used in one or more of the following other applications. In one example, process 200 may be used in medical imaging. For example, an image of an injury (e.g., broken leg, severed finger and so forth) is uploaded to the image analyzer 112. The image analyzer 112 will interpret the image and determine severity of the injury. The image analyzer 112 may send the information to the proper emergency services and/or deliver medical advice on how to address the injury. For example, if the image analyzer 112 interprets the image as including a severed finger, the user can be sent instructions for preserving the severed portion of the finger for re-attachment.

In another example, an image of a meal is uploaded to the image analyzer 112. The image analyzer 112 identifies the type of food and provides from one or more of the services 120 at least one of an estimate of the calories in the meal, ties in medical advice and/or medical costs of consuming the meal. In one embodiment, a user can upload an image for each meal for analysis by the image analyzer 112 in exchange for a potential discount on medical insurance.

In another example, an image of food is uploaded to the image analyzer 112. The image analyzer 112 identifies the type of food and provides, from one or more of the services 120, food allergy detection and/or delivers likely ingredients for allergies and food analysis.

In another example, an image of meat is uploaded to the image analyzer 112. The image analyzer 112 identifies the meat and determines, from one or more of the services 120, the doneness of meat from the color of the meat and suggests how to better cook the meat.

In another example, an image of a shopping cart of objects is uploaded to the image analyzer 112. The image analyzer 112 may provide from one or more of the services 120 an estimate of the monetary value of the objects in the shopping cart and/or may tie the monetary value of the objects with a budget.

In a further example, an image of a house is uploaded to the image analyzer 112. The image analyzer 112 may provide from one or more of the services 120 an estimate of the cost to insure and maintain the house.

In a further example, an image of a defect in a house is uploaded to the image analyzer 112. The image analyzer 112 may provide from one or more of the services 120 an estimate on a price and how to fix the defect. For example, an image of a house can be analyzed to determine whether new roof shingles, windows, siding, should be replaced to prevent damage to a structure. If, for example, the image analyzer determines that roof shingle are in a condition to incur water leaks in the roof, a property owner can be contacted with an estimate and potential roofer to replace or repair the shingles.

In a further example, an image of a machine is uploaded to the image analyzer 112. The image analyzer 112 may provide from one or more of the services 120 a description or images on how the machine works and/or delivering advice and questions to triage a situation. Example machines include consumer goods, like radios, home appliances, electronics and cars and car engines, as well as industrial machinery like robots, reactors, generators and the like.

In a further example, an image of several objects is uploaded to the image analyzer 112. The image analyzer 112 may provide from one or more of the services 120 a number for each object from smallest to largest size or visa-versa, cheapest to most expensive or visa-versa, oldest to newest or visa-versa, and so forth.

In a further example, an image of an object is uploaded to the image analyzer 112. The image analyzer 112 may provide from one or more of the services 120 a space estimation to estimate where the object might fit within a space. In embodiments, space estimation can determine room dimensions for decoration or crowd control, the size of an object to fit in a designated space, such as buying furniture to see if it will fit in a chosen position, and determining if a new building would fit in an undeveloped field.

In a further example, an image of an object is uploaded to the image analyzer 112. The image analyzer 112 may provide, from one or more of the services 120, a weight estimation to estimate the relative weight of objects and suggest a cheapest way to ship, lift and/or validate size.

In another example, an image from a camera is provided to the image analyzer 112, which identifies plants and animals in the image. Metadata for the image can include beacons and geolocation to increase the accuracy of the determination of what is in the image. For example, geolocation may be useful in public gardens and zoos.

In a further example, an image from a camera is provided to the image analyzer 112, which identifies a monument to enable delivery of information about the monument in the image. Beacon and/or geolocation and the like can enhance image recognition of the monuments. Other object can include historical markers, museums, cemeteries and the like to obtain and provide additional information via text, video, images etc.

In still further examples, the system 100 may be used as a digital notary by taking a video of a signature after biometric sign in and adding a notary stamp on a screen and validating that the signature verifies with earlier signature samples.

In another example, the system 100 may be used be for multiple signatures at the same time (e.g., co-signers). For example, the co-signers hold up their mobile devices at the same time, use biometrics to digitally sign the same document at the same time and send in the document as one image.

In another example, the system 100 may be used be to automatically transcribe music. For example, an image of an instrument is sent, as it is played. The music is delivered on screen. In another example, an image of a sheet of music is taken and the music appears on a screen in a different/desired key.

In another example, the system 100 may be used be for digital homework submission. For example, a picture of homework, such as multiple choice questions, is taken and is graded immediately. In another example, the system 100 delivers how to finish the answer to a problem correctly on a screen.

In still further examples, system 100 may be used to inventory a home by taking images of the objects in the home for insurance or to be used for a will to bequeath objects to certain individuals or organizations.

In embodiments, delivery of a service can be provided through a digital user interface, for example, that enables a user to take and upload photos, video or live video stream. Information about the identified object(s) can be presented on the screen to provide information about the object and allow the user to take one or more actions. If the user decides to take action and/or verify the object presented as being of interest, then another service can be called and the desired user action can be performed.

In one embodiment in associating with preparation of a will, for example, individual objects identified in the image of the objects can be identified to allow the user to make selections for inclusion in a will. The service can invite the user to choose a person to whom they would like to give the selected object. From this data, a generic template for a will provided by the service, can be filled in with the captured information. The user can be prompted to review and accept the will. In embodiments, the user can digitally sign the document, or print it and sign it using an image of the document with a signature, as described above, and then given the ability to upload or send the document to a desired location. In other instances, such as for insurance purposes, the user can begin an application for insurance and skip some of the steps required to fill in a complete insurance application. In other forms, the user is provided information about objects that they can use to fix, address or gain training, like in the instance of medical applications, how machines work, and homework grading.

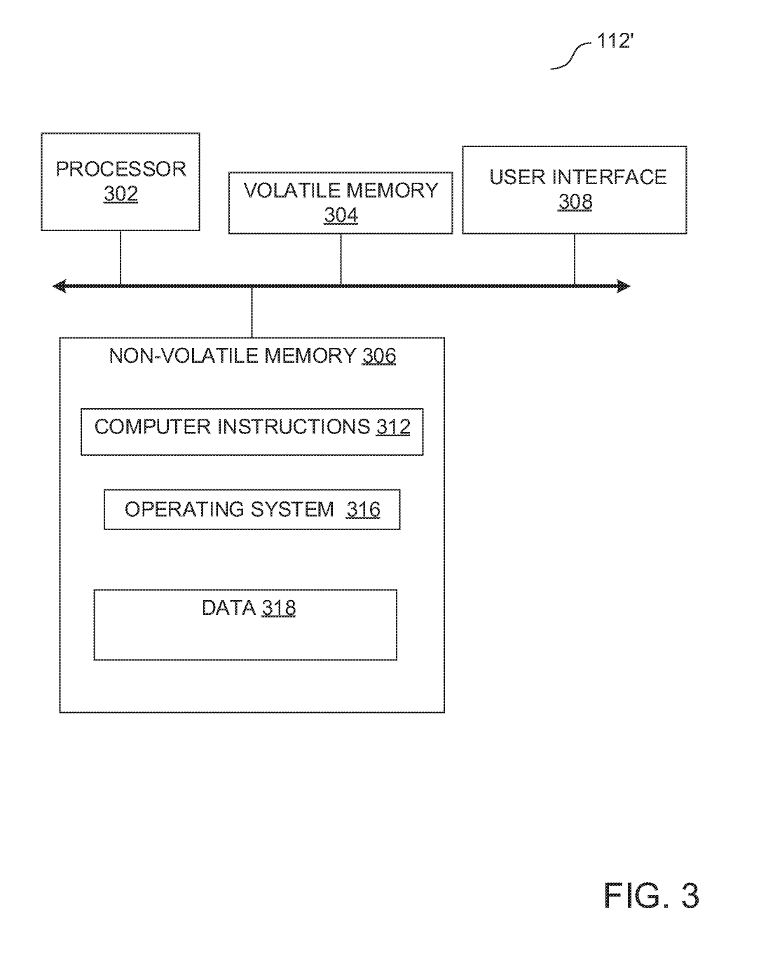

Referring to FIG. 3, in one example, the image analyzer 112 is an image analyzer 112′. The image analyzer 112′ may include a processor 302, a volatile memory 304, a non-volatile memory 306 (e.g., hard disk, flash memory) and the user interface (UI) 308 (e.g., a graphical user interface, a mouse, a keyboard, a display, touch screen and so forth). The non-volatile memory 306 may store computer instructions 312, an operating system 316 and data 318. In one example, the computer instructions 312 may be executed by the processor 302 out of volatile memory 304 to perform at least a portion of the processes described herein (e.g., process 200).

The processes described herein (e.g., process 200) are not limited to use with the hardware and software of FIG. 3; they may find applicability in any computing or processing environment and with any type of machine or set of machines that can run a computer program. The processes described herein may be implemented in hardware, software, or a combination of the two. The processes described herein may be implemented in computer programs executed on programmable computers/machines that each includes a processor, a non-transitory machine-readable medium or other article of manufacture that is readable by the processor (including volatile and non-volatile memory and/or storage elements), at least one input device, and one or more output devices. Program code may be applied to data entered using an input device to perform any of the processes described herein and to generate output information.

The system may be implemented, at least in part, via a computer program product, (e.g., in a non-transitory machine-readable storage medium such as, for example, a non-transitory computer-readable medium), for execution by, or to control the operation of, data processing apparatus (e.g., a programmable processor, a computer, or multiple computers)). Each such program may be implemented in a high level procedural or object-oriented programming language to communicate with a computer system. However, the programs may be implemented in assembly or machine language. The language may be a compiled or an interpreted language and it may be deployed in any form, including as a stand-alone program or as a module, component, subroutine, or other unit suitable for use in a computing environment. A computer program may be deployed to be executed on one computer or on multiple computers at one site or distributed across multiple sites and interconnected by a communication network. A computer program may be stored on a non-transitory machine-readable medium that is readable by a general or special purpose programmable computer for configuring and operating the computer when the non-transitory machine-readable medium is read by the computer to perform the processes described herein. For example, the processes described herein may also be implemented as a non-transitory machine-readable storage medium, configured with a computer program, where upon execution, instructions in the computer program cause the computer to operate in accordance with the processes. A non-transitory machine-readable medium may include but is not limited to a hard drive, compact disc, flash memory, non-volatile memory, volatile memory, magnetic diskette and so forth but does not include a transitory signal per se.

The processes described herein are not limited to the specific examples described. For example, the process 200 is not limited to the specific processing order of FIG. 2. Rather, any of the processing blocks of FIG. 2 may be re-ordered, combined or removed, performed in parallel or in serial, as necessary, to achieve the results set forth above.

The processing blocks (for example, in the process 200) associated with implementing the system may be performed by one or more programmable processors executing one or more computer programs to perform the functions of the system. All or part of the system may be implemented as, special purpose logic circuitry (e.g., an FPGA (field-programmable gate array) and/or an ASIC (application-specific integrated circuit)). All or part of the system may be implemented using electronic hardware circuitry that include electronic devices such as, for example, at least one of a processor, a memory, a programmable logic device or a logic gate.

Elements of different embodiments described herein may be combined to form other embodiments not specifically set forth above. Various elements, which are described in the context of a single embodiment, may also be provided separately or in any suitable subcombination. Other embodiments not specifically described herein are also within the scope of the following claims.

What is claimed is:

1. A method comprising:
receiving an image;
processing the image to identify one or more objects in the image; and
selecting a service based on the one or more objects identified in the image.

2. The method of claim 1, wherein the image is a photo.

3. The method of claim 1, wherein the image is a video.

4. The method according to claim 1, wherein the one or more objects includes a person and the service is selected based upon an age of the person.

5. The method according to claim 1, wherein the one or more objects includes at least two people and the service is selected based upon an identified relationship between the at least two people.

6. The method according to claim 1, wherein the one or more objects includes a person with a disability and the service is selected based upon the disability.

7. The method according to claim 1, wherein the one or more objects includes medical injury and the service is selected based upon the injury.

8. The method according to claim 1, wherein the one or more objects includes food and the service is selected based upon a type of the food.

9. The method according to claim 1, wherein the one or more objects includes food and the service is selected based upon a type of the food for food allergy detection.

10. The method according to claim 1, wherein the one or more objects includes cooked meat and the service is selected based upon an identified doneness of the meat.

11. The method according to claim 1, wherein the one or more objects includes a structure and the service is selected based damage to the structure.

12. The method according to claim 1, wherein the one or more objects includes a structure and the service is selected based upon maintenance needed for the structure.

13. The method according to claim 1, wherein the one or more objects includes a structure and the service is selected based upon maintenance needed for the structure, wherein the service includes a cost estimate for the needed maintenance.

14. The method according to claim 1, wherein the service includes shipping of the one or more objects and the service is selected based upon an estimate weight of the one or more objects.

15. The method according to claim 1, wherein the service includes shipping of the one or more objects and the service is selected based upon an estimate weight of the one or more objects, and the service includes a cost estimate for shipping of the one or more objects.

16. The method according to claim 1, wherein the one or more objects includes objects in a home and the service includes insurance services for protecting against loss or damage to the one or more objects.

17. A system, comprising:
an image module to receive an image;
an image analyzer module for processing the image to identify one or more objects in the image; and
a service module coupled to the image analyzer module for selecting a service based on the one or more objects identified in the image.
